(19) United States
(12) Patent Application Publication
(10) Pub. No.: US 2021/0299002 A1
(43) Pub. Date: Sep. 30, 2021

(54) DURABLE ABSORBENT WIPER

(71) Applicant: Kimberly-Clark Worldwide, Inc., Neenah, WI (US)

(72) Inventors: Allen F. Vater, Alpharetta, GA (US); Bryan D. Haynes, Cumming, GA (US); Alejandro Romero Villarreal, Buford, GA (US); Michelle Hernandez, Roswell, GA (US); Lauren Munoz, Atlanta, GA (US)

(21) Appl. No.: 16/327,136

(22) PCT Filed: Aug. 30, 2017
(86) PCT No.: PCT/US17/49396

§ 371 Date: Feb. 21, 2019

Related U.S. Application Data
(60) Provisional application No. 62/381,830, filed on Aug. 31, 2016.

(51) Int. Cl. A61K 8/02; A61Q 19/10; A61K 8/81; A61K 8/73; D04H 1/56; D04H 1/4291; D04H 3/16; D04H 3/007; D04H 1/407; D04H 5/00; A47K 10/42; A47K 10/16

(52) U.S. Cl. CPC A61K 8/0208; A47K 2010/3266; A61K 8/8105; A61K 8/731; D04H 1/56; D04H 1/4291; D04H 3/16; D04H 3/007; D04H 1/407; D04H 5/00; A47K 10/421; A47K 10/16; A61K 2800/87; D10B 2509/026; A61Q 19/10

(57) ABSTRACT

Absorbent nonwoven wipes having improved strength and hand feel comprising a non-homogeneous mixture of small diameter polymeric meltblown fibers and absorbent staple fibers with meltblown fiber rich outer surfaces and staple fiber rich inner regions and wherein a minor amount of staple fibers also protrude through the meltblown fiber rich regions. Wet wipes and dispensers for housing the same are also provided.

### FIELD OF INVENTION

The present invention relates to disposable cleaning articles including a mixture of polymeric meltblown fibers and absorbent fibers for use as wipers in personal care applications.

### BACKGROUND

Disposable wipers, including wet wipes, have long been used in a number of different personal care and hygiene related applications including, for example, as perineal wipes, hand wipes, face wipes, and so forth. Users of such wipes desire a wipe that has a soft and pleasing feel to the touch and that also effectively cleans the skin. In addition, users also desire a wipe that resists tearing, linting or the generation of any unwanted debris when used. Further, users also desire a wipe that is consistently moist having neither too much nor too little moisture. In this regard, in order to maintain the desired moisture content, it is common for multiple wet wipes to be housed in a resealable dispenser and removed therefrom only immediately prior to use. However, both converting and dispensing of the wipes puts torsional and other forces on the wipe that can result in tearing, fraying or other damage to the wipe. Achieving the desired tactile properties and cleaning efficacy together with the desired strength and absorbency needed for converting, dispensing and use has proven difficult. In this regard, softness, strength and absorbency are often competing properties in that increasing one can negatively impact one or more of the other properties.

Therefore, in order to address the unmet needs associated with prior wet wipes, the present invention provides wet wipes having a unique and improved combination of properties, namely pleasing hand together with high levels of cleaning efficacy, absorbency and tear resistance. Such wipes are stronger and more durable, being better able to withstand the forces and stresses commonly associated with converting, dispensing and/or use, and yet which simultaneously provide other competing functional properties important to the user.

### SUMMARY OF THE INVENTION

The present invention provides a wipe comprising a nonwoven web having a non-homogeneous mixture of between about 20-40 wt. % polymeric meltblown fibers and about 60-80% absorbent staple length fibers. The polymeric meltblown fibers of the nonwoven web comprise a propylene polymer and also have an average fiber size between 1.0-3.0 micrometers and a melt-flow rate between about 200 and about 950 dg/minute. Further, the polymeric meltblown fibers comprise the majority of fibers forming the major outer surfaces of the web and the absorbent staple length fibers present within the major outer surfaces protrude therefrom and/or are unoccluded by the polymeric meltblown fibers forming the major outer surfaces. Still further, despite having a relatively uniform outer layer of fine meltblown fibers, the nonwoven web is configured to have an air permeability between about 50 and about 300 CFM and a cross-direction tensile strength of at least about 190 grams-force. In addition, impregnated within the nonwoven wipe is a liquid cleaning formulation, comprising water and a surfactant, in an amount between about 50% to about 700% based upon the weight of the dry nonwoven web.

In certain embodiments, the staple length fibers comprise cellulosic fibers such as, for example, wood pulp fibers. In further embodiments, the propylene polymer can comprise a blend of one or more different propylene polymers and, additionally or alternatively, comprise greater than about 85% of the polymeric meltblown fibers. The nonwoven web presents a pleasing hand and in certain aspects the major outer surfaces of the nonwoven web have a TS7 softness rating less than 7.0. In still further embodiments, the nonwoven web can have a basis weight of between about 30-90 g/m² and a tensile strength between about 190 and about 300 g-f.

### BRIEF DESCRIPTION OF THE DRAWINGS

FIG. 1 is a perspective view of a wipe of the present invention.

FIG. 2A is an SEM photomicrograph of a cross-sectional view of a nonwoven wipe of the present invention.

FIG. 2B is an SEM photomicrograph of a top surface of a nonwoven wipe of the present invention.

FIG. 3 is perspective view of a stack of wipes of the present invention.

FIG. 4 is a perspective, partially cut-away view of an open dispenser having a stack of wipes of the present invention.

FIG. 5 is a side, perspective view an open tub style dispenser suitable for use in dispensing a stack of wipes of the present invention.

FIG. 6 is a side, perspective view an open flexible pack dispenser having a stack of wipes of the present invention.

### DETAILED DESCRIPTION

Throughout the specification and claims, discussion of the articles and/or individual components thereof is with the understanding set forth below.

(i) The term “comprising” or “including” or “having” are inclusive or open-ended and do not exclude additional unrecited elements, compositional components, or method steps. Accordingly, the terms “comprising” or “including” or “having” encompass the more restrictive terms “consisting essentially of” and “consisting of.”

(ii) As used herein “continuous fibers” means fibers formed in a substantially continuous, uninterrupted manner having indefinite length and having a high aspect ratio (length to diameter) in excess of about 10,000:1; in use the majority of such fibers can having a length substantially the same as the length or width dimension of the wiper itself.

(iii) As used herein “staple length fibers” means continuous synthetic fibers cut to length or natural fibers, such fibers having a length between about 0.5 mm and about 90 mm. The length of such fibers being that of the straight (e.g. uncontorted) fiber.

(iv) As used herein, unless expressly indicated otherwise, when used in relation to material compositions the terms “percent” or “percent” each refer to the quantity by weight of a component as a percentage of the total.

(v) As used herein the term “cellulosic” means those materials comprising or derived from cellulose including natural or synthetic cellulose as well as that derived from both woody and non-woody sources.

(vii) As used herein, the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

(vii) As used herein “propylene polymer” means a polymer having greater than 50% propylene content.

(viii) As used herein, the term “nonwoven web” means a structure or a web of material that has been formed without use of traditional fabric forming processes such as weaving or knitting, to produce a structure of individual fibers or threads that are entangled or intermeshed, but not in an identifiable, repeating manner.

(ix) As used herein, the term “machine direction” or “MD” refers to the direction of travel of the forming surface onto which fibers are deposited during formation of a fibrous web.

(x) As used herein, the term “cross-machine direction” or “CD” refers to the direction which is essentially perpendicular to the machine direction defined above.

(xi) As used herein, the term “emboss” or “embossment” means a substantially permanent localized depression in the sheet.

(xii) As used herein, the terms “TS7” and “TS7 value” refer to an output of an EMTEC Tissue Softness Analyzer (“ISA”) (Emtec Electronic GmbH, Leipzig, Germany) as described in the Test Methods section. The units of the TS7 value are dB V² rms, however, TS7 values are often referred to herein without reference to units.

(xii) As used herein, the term “stack” is used broadly to include any collection of sheets wherein there is a plurality of individual sheets having surface-to-surface interfaces; this not only includes a vertically stacked collection of individual sheets, but also includes a horizontally stacked collection of individual sheets as well as rolled or folded collection of sheets.

### Nonwoven Cleaning Sheet

Absorbent nonwoven cleaning articles of the present invention have a pleasing hand, excellent strength, absorbency and high cleaning efficacy and are provided for use in one or more various personal care and/or hygienic cleaning tasks. The nonwoven web comprises a porous material having numerous individual openings or interstitial spaces which collectively form pathways through the thickness of the material via adjacent, inter-connecting spaces or openings. The nonwoven web retains its integrity when wet and, in certain aspects, remains resiliently compressible when wet. The cleaning articles of the present invention include a nonwoven web comprising a particular combination of polymeric meltblown fibers and absorbent staple length fibers such as cellulosic fibers.

The pleasing hand and/or softness is achieved at least in part by a nonwoven web having a relatively uniform surface layer of fine meltblown fibers. In this regard, the meltblown fibers of the nonwoven web will have an average fiber diameter of between 1.0 and 3.0 micrometers. In certain embodiments, the average fiber diameter of the meltblown fibers may be greater than 1.0, 1.2, 1.3, 1.4, 1.5 or 1.6 and/or the average fiber diameter of the meltblown fibers may be less than 3.0, 2.9, 2.8, 2.7 or 2.6. Additionally, the meltblown fibers may have a melt-flow rate (MFR) of between about 200 and about 950 dg/min. In certain embodiments the MFR can be greater than about 250, about 350 or about 400 dg/min. and/or may be less than about 950, about 900, about 850 or even about 800 dg/min.

The polymeric meltblown fibers may be continuous or substantially continuous. As used herein “substantially continuous” means fibers having an average fiber length of greater than about 100 mm. The meltblown fibers may be formed according to one or more various processes including, but not limited to, those described in U.S. Pat. No. 3,849,241 to Butin et al., U.S. Pat. No. 6,972,104 to Haynes et al. and U.S. Pat. No. 8,017,534 to Harvey et al. With respect to the meltblowing process, processing conditions are selected in relation to the particular equipment and polymer utilized in order to achieve the desired fiber size and web structure. Typically, however, the size of the orifices used to extrude the molten polymer from the die orifice can be between about 0.2 and about 0.4 mm and can have a spacing of about 10-40 per cm or from about 14-30 per cm. In addition, the molten polymer can be pumped through the orifices at a pressure of between about 12 Kg-f/cm² (175 PSI) and about 70 Kg-f/cm² (1000 PSI). Further, the draw air can have a temperature between about 200° C. and about 315° C. or between about 230° C. and about 290° C. and may be applied at a pressure of between about 0.2 Kg-F/cm² (3 PSI) and about 0.6 Kg-f/cm² (9 PSI).

Polymers suitable for forming fibers capable of providing the unique properties discussed herein include propylene polymers having an MFR less than about 850 dg/min. In certain embodiments, the propylene polymer has an MFR of less than about 800, about 750 or even less than about 700 dg/min. and/or has an MFR greater than about 25, about 150, about 250 or even greater than about 300 dg/min. Propylene polymers having relatively lower MFRs may be used in conjunction with peroxide cracking or other known means for increasing the melt-flow rate in association with the melt-extrusion process. The polymer comprising the meltblown fibers can comprise more than one propylene polymer, however the resulting MFR of the formed fibers should still reside within the ranges described herein. In certain embodiments, the polymeric portion of the meltblown fibers can comprise between about 85-100%, 90-100% or even between about 95-100% propylene polymer. Suitable propylene polymers can be made by one or more ways known in the art including, for example, those made by either Ziegler-Natta, metallocene, single-site and chromium catalysts. The propylene polymer may be a homo-polymer or a mix such as, for example, a polymer of propylene together with one or more other monomers such as, for example, linear and branched alpha olefins. Exemplary co-monomers include, but are not limited to, 1-pentene, 1-hexene, 1-heptene, 1-octene, 1-butene, 4-methyle-1-pentene, 3-methyle-1-pentene, 1,7-octadiene, 1,9-decadiene, and so forth. The propylene polymer comprises at least 50% propylene units and in certain embodiments may comprise greater than 60%, 70%, 80% or even 90% propylene units. As an example, commercially available propylene polymers suitable for use in the present invention include, but are not limited to, MF650W available from PolyMirae Company LTD; MF650W available from Lyondell Bassell Industries and ACHIEVE 6305G1 from Exxon Mobile Corporation.

In addition, the polymer blend prepared to form, and the resulting meltblown fibers, may include a minor portion of one or more additives such as, for example, additional compatible polymers (including non-propylene polymers), stabilizers, tackifiers, surfactants, pigments, dyes, fragrances, viscosity modifiers, fillers and so forth. The use of such additional components are well known in the art. Such additional components desirably comprise less than about 15%, 10%, 5% or even 3% of the meltblown fibers. In one aspect the polymers used to form the meltblown fiber web may also include a surfactant such as, for example, TECHSURF PPM 15560 available from Techmer PM.

As noted above, the nonwoven webs also include staple length absorbent fibers such as, for example, cellulosic fibers. The cellulosic fibers may comprise traditional paper making fibers including woody fibers such as those obtained from deciduous and coniferous trees, including, but not limited to, softwood fibers, such as pine, firm, and spruce, and also hardwood fibers, such as eucalyptus, maple, birch, and aspen. Other papermaking fibers that can be used in the present invention include paper broke or recycled fibers and high yield fibers. Various pulping processes believed suitable for the production of cellulosic fibers include bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), high yield sulfite pulps, and high yield Kraft pulps. Debonded fluff pulps are particularly well suited for use in the present invention. In addition, the cellulosic fibers may comprises non-woody fibers, such as cotton, abaca, bamboo, kenaf, sabai grass, flax, esparto grass, straw, jute hemp, bagasse, milkweed floss fibers, pineapple leaf fibers and so forth. Still further, the cellulosic fibers may comprise synthetic fibers derived from cellulosic materials such as viscose, Rayon, lyocell or other comparable fibers. Moreover, if desired, secondary fibers obtained from recycled materials may be used, such as fiber pulp reclaimed from sources such as, for example, newsprint, paperboard, office waste, etc. The fibrous sheet material can comprise a single variety of cellulosic fibers or alternatively can comprise a mixture of two or more different cellulosic fibers. As is known in the art, it is often desirable to employ mixtures of fibers especially when utilizing recycled or secondary fibers.

The nonwoven web includes at least 20% meltblown fibers and at least 60% absorbent staple length fibers. Apparatus and methods for co-mingling meltblown and staple-length fiber streams are described in U.S. Pat. No. 5,350,624 to Georger et al., U.S. Pat. No. 6,972,104 to Harvey et al and U.S. Pat. No. 9,260,808 to Schmidt et al. In certain embodiments, the nonwoven web can include at least about 25%, 27% or even 30% meltblown fibers and/or less than about 40%, 37% or even 35% meltblown fibers. Further, in certain embodiments, the nonwoven web can include at least about 60%, 63%, or 65% staple length absorbent fibers and/or less than about 80%, 75%, 73% or even 70% staple length absorbent fibers. In preferred embodiments, the meltblown fibers are predominantly at and adjacent the major web surfaces. In other words, the meltblown fibers desirably are disproportionately located towards the outer surfaces of the nonwoven web, creating a meltblown fiber rich surface. In this regard, in order to achieve nonwoven webs having the desired fiber distribution, the meltblown fibers may be directed to impinge upon the flow of cellulosic fibers at an impingement angle of between about 40-55 degrees and more desirably between about 45 and about 50 degrees. However, it is necessary for the wipes to retain the ability to take-up and release liquids, therefore the nonwoven web requires a sufficiently porous and non-homogeneous surface structure.

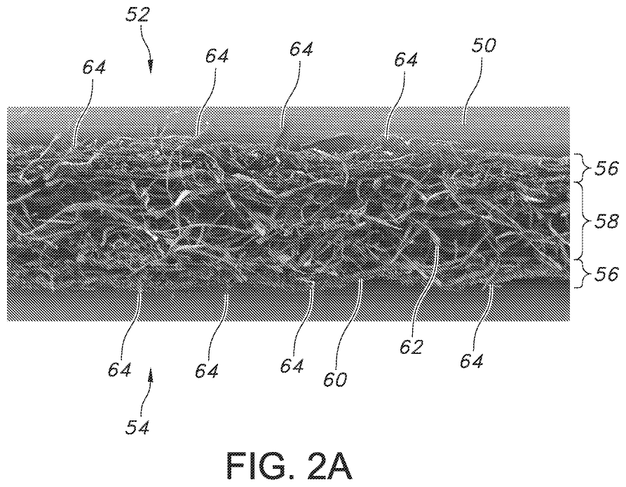

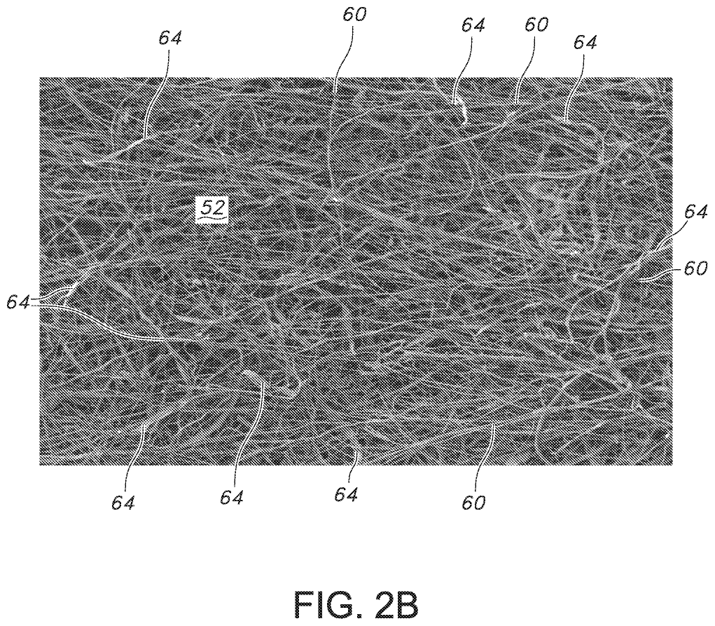

While the use of smaller diameter fibers such as used herein are commonly employed in barrier grade products such as in filters or medical products, it has been found that the controlled and selective use of such fibers allows for the formation of a nonwoven web having the required durability and liquid handling properties and yet which also achieves desired softness, absorbency and good cleaning efficacy. In this regard, it is important that the fiber distribution and orientation should be such that the major outer surfaces have a significant number of absorbent fibers that are unoccluded by the meltblown fibers and/or exposed on the outer surface of the web. As seen in reference to FIGS. 2A and 2B, the composite nonwoven web 50 includes a first major outer surface 52, second major outer surface 54 and also having meltblown rich regions 56 and a pulp rich region 58. The majority of pulp fibers 62 are unexposed and contained entirely within or between the meltblown fiber layers. The meltblown rich regions 56 form the outer surfaces 52, 54 and while the meltblown fibers 60 are predominantly located at or adjacent the major outer surfaces 52, 54, a significant number of pulp fibers 64 are contained within and/or protrude through the meltblown fiber rich region 56 and outer surfaces 52, 54 surface thereby achieving a structure and orientation of fibers that is sufficiently porous and inclusive of absorbent fibers so as to allow for the passage of liquids there through. Despite the use of finer small diameter meltblown fibers typically associated with barrier function, suitable liquid handling properties can be achieved with this structure. With respect to porosity, one measure of porosity is air-flow; a more open and/or porous web will more readily allow air to pass there through. Nonwoven webs of the present invention are configured to present a sufficiently porous structure to have an air permeability greater than about 50 CFM (about 1.4 m³/min.) and in certain embodiments can have an air permeability greater than about 70 CFM (about 2 m³/min.) or even 90 CFM (about 2.5 m³/min.). Further, in certain embodiments, the nonwoven web can an air permeability less than about 300 CFM (8.5 m³/min.), 250 CFM (about 7 m³/min.), 225 CFM (about 6.5 m³/min.), 200 CFM (about 5.7 m³/min.) or even 180 CFM (about 5 m³/min.).

The nonwoven webs will typically have a dry basis weight of from about 20 g/m² to about 260 g/m². In certain embodiments, the dry basis weight of the fibrous sheet will be between about 30 g/m² to about 190 g/m² and in still further embodiments may be between about 30 g/m² to about 120 g/m². The attributes of the nonwoven web of the present invention are particularly well suited for use with lower basis weight materials including, for example, those having a basis weight between about 20 g/m² and about 90 g/m² or between about 30 g/m² and about 80 g/m² or even between about 35 g/m² and about 70 g/m². In addition, the nonwoven web will have a CD tensile strength of at least about 190 g-f. In certain embodiments the nonwoven web will have a tensile strength greater than about 190 g-f, 200 g-f, 210 g-f or even 220 g-f. In addition, in certain embodiments the nonwoven web will have a tensile strength less than about 400 g-f or even less than about 350 or about 300 g-f. In still further embodiments, the nonwoven web may have a tensile strength, normalized for basis weight, of at least about 15 g-f per g/m² such as, for example being at least about 15, 16 or even 17 b g-f per g/m² and/or less than about 25, 24 or 22 g-f per g/m². Despite the excellent strength, the nonwoven web continues to present a pleasing hand and/or soft feel. In this regard, the nonwoven web will, despite its strength, have a TS7 value of between about 4.0 and about 7.0, between about 4.5 and 6.5, between about 5.0 and 6.5, or even between about 5.0 and 6.0.

In a further aspect, the meltblown fiber portion of the nonwoven web is formed with a relatively higher degree of uniformity as compared to like absorbent nonwoven wipes. In this regard, the meltblown fibers can have a Formation Index of between about 20.0 and about 10.0 or even between about 12.0 and about 16.0.

Optionally, the nonwoven web may be treated in one or more additional ways as desired. For example, surfactants may be applied to the web in order to improve the ease with which water penetrates the web. Additionally and/or alternatively, the nonwoven web may be treated to impart aesthetically pleasing and/or texture enhancing patterns to the nonwoven web. For example, the nonwoven web may be treated by one or more embossing techniques known in the art that impart localized compression and/or bonding corresponding to one or more desired patterns. In this regard, the base sheet can be embossed by the application of localized pressure, heat, and/or ultrasonic energy. In certain aspects, the base sheet may be embossed as is known in the art by using a pair of embossing rolls, wherein at least one of the rolls has a pattern of protuberances or “pins” corresponding to the desired pattern of emboss elements to be imparted to the base sheet. The two cooperative rolls form a nip through which the base sheet is passed with the application of pressure and, optionally, heat. While suitable embossments may be formed without the application of heat, use of heat together with pressure is preferred. The embossing can be conducted as is known in the art employing a nip formed by patterned roll and a smooth anvil roll (“pin-to-flat”) or by two coordinated patterned rolls (“pin-to-pin”). With respect to the use of a smooth anvil roll, such roll may be coated with a resilient material, such as rubber, in order to improve the formation of embossments within the web. By way of example only, various embossing methods are shown and described in U.S. Pat. No. 3,855,046 to Hansen et al., U.S. Pat. No. 5,620,779 issued to Levy et al., U.S. Pat. No. 6,036,909 to Baum, U.S. Pat. No. 6,165,298 to Samida et al. and U.S. Pat. No. 7,252,870 to Anderson et al. The sheet materials may be embossed and/or bonded by continuous and/or discontinuous lines, by patterns of numerous discrete elements, or other patterns as may be desired. The embossments can comprise linear, curvilinear, geometric or even iconic elements such as for example a cloud, dog, bear, leaf, feather, flower or other recognizable image or character. Further, the nonwoven web may be embossed uniformly across the surface or regionally such as along the periphery or edge of the sheet. The total embossed area will generally be less than about 50% of the surface area of the nonwoven web and more desirably will be between about 2% and about 30% of the web or even between about 4% and about 20% of the web. As further options, the nonwoven webs may, additionally or alternatively, be treated by various other known techniques such as, for example, stretching, needling, creping, and so forth. Still further, the nonwoven web may optionally be plied with one or more additional materials or fabrics.

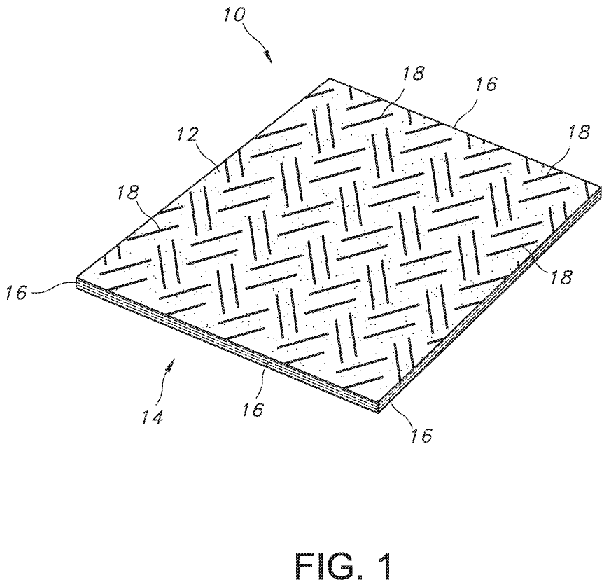

In reference to FIG. 1, a wiper 10 is provided defined by first major surface or upper surface 12 and an opposed second major surface or bottom surface 14 extending parallel thereto. Extending between the upper and bottom surfaces 12, 14 are one or more minor surfaces or edges 16. The dimensions of the wiper can vary in accordance with the particular end use and/or desired function of the wiper. In certain embodiments, the wiper can have a diameter (in its greatest dimension) of between about 5 and about 45 cm, and in certain embodiments between about 10 cm and about 30 cm. In other embodiments, the wipe can have a length (extending in the direction of the longer dimension) between about 5 cm and about 30 cm or between about 12 cm and about 25 cm. Further, the wiper can have a width (extending in the direction perpendicular to the lengthwise direction) between about 5 cm and about 30 cm or between about 10 cm and about 20 cm. The wiper can have any one of various shapes such as rectangular, square, elliptical, round and so forth. In addition, the edges themselves may be cut to have a straight edge or to have a more complex or irregular shape such as being curvilinear (e.g. having a scalloped or sinusoidal shaped edge). Still in reference to FIG. 1, the nonwoven wipe 10 may optionally include a plurality of embossments therein such as, for example, a repeating pattern of discrete embossments 18.

### Liquid Cleaning Formulations

A liquid cleaning composition can be incorporated into the nonwoven web to from a wet wipe. Desirably, the liquid cleaning composition is added to and impregnated within the base sheet in an amount of from about 50% to about 700% (by weight of the dry nonwoven web), more desirably from about 75% to about 500%, even more desirably from about 100% to about 400% or from about 100% to about 350%. A wide variety of liquid cleaning formulations are believed suitable for use with the present invention. The selection of a particular cleaning formulation will vary significantly with the intended end use of the wiper and other factors known to those skilled in the art. The liquid cleaning composition can comprise a solution, emulsion or dispersion. By way of example, cleaning compositions believed suitable for use with the present invention include, but are not limited to, those described in U.S. Pat. No. 4,772,501 Johnson et al., U.S. Pat. No. 4,941,995 Richards, U.S. Pat. No. 8,563,017 Cunningham et al., U.S. Pat. No. 8,987,180 Wenzel et al., US2010/0256033 to Menard et al. and WO2015/084880 to Park et al.

The liquid cleansing composition includes at least a solvent and a surfactant. The solvent can comprise water, alcohol (e.g. IPA) or mixtures of water and alcohol. Desirably, the cleaning formulation comprises an aqueous formulation comprising greater than 50% water and still more desirably at least about 70% water. In certain embodiments, the cleansing formulation can include between about 80% and about 99.5% water or between about 85% and about 99% water or even between about 90% and about 98% water. In many applications, such as with respect to perineal wipes, the use of higher percentages of water can help reduce skin sensitivity and/or help prevent the user from experiencing a sticky or tacky feeling after use.

As noted above, the liquid cleaning composition includes one or more surfactants. The cleaning composition may comprise less than about 20% surfactant, based upon the weight of the liquid cleaning composition. In certain embodiments, the surfactant can comprise less than about 15% or less than about 10% of the cleaning composition. In certain embodiments, the cleaning formulation can comprise between about 20% and about 0.5% surfactant, or between about 15% and 1% surfactant or even between about 10% and about 2% surfactant. Numerous surfactants are believed suitable for use with the present invention including non-ionic surfactants, anionic, cationic, amphoteric, zwitterionic and combinations thereof.

In certain embodiments, the surfactant will comprise one or more nonionic surfactants. By way of examples, suitable classes of nonionic surfactants include, but are not limited to, alkyl glycosides and alkyl polyglycosides, alkyl glucosides and alkyl polyglucosides, ethoxylated alkylphenols, ethoxylated fatty (C₈-C₂₂) alcohols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, and mixtures thereof. One or more anionic, cationic, or zwitterionic surfactants may also be used in the cleaning composition of the present invention, either alone or in combination with other surfactants. By way of example, additional surfactants believed suitable for use in the present invention includes, but are not limited to, alkyl sulfates, alkyl ether sulfates, alkyl ether sulfonates, alkylauryl sulfonates, fatty acid amide polyoxyethylene sulfates, aminopropyl alkylglutamide, sodium alkyliminodipropionate, carboxybetaines, sulfobetaines, phosphobetaines, phosphitaines, amine oxides, cocamidopropyl betaine, coco-betaine; lauryl hydroxy sulfobetaines, alkyl amines, dimethyl alkyl amines, alkyl betaines, quaternized APG, ethoxylated alkylamines, and combinations thereof.

The liquid cleaning composition may, optionally, include one or more preservatives or antimicrobial agents to increase the shelf life of the wet wipe. Suitable preservatives that can be used in the present invention include, but are not limited to, sodium and other metal salts of benzoic acid (e.g. sodium benzoate available under the trade name PUROX S from Emerald Performance Materials); mixtures of methylchloroisothiazolinone and methylisothiazolinone (e.g. KATHON CG from Dow Chemical); methylisothiazolinone (e.g. NELONE 950 from Rohm Haas); DMDM hydantoin and iodopropynyl butylcarbamate (e.g. GLYDANT PLUS from Lonza); hydroxybenzoic acid esters (parabens), such as methylparaben, propylparaben, butylparaben, ethylparaben, benzylparaben, sodium methylparaben, and sodium propylparaben; 2-bromo-2-nitropropane-1,3-diol (e.g. Bronopol from BASF); benzoic acid; sorbic acid and its salts; amidazolidinyl urea (e.g. Germall 115 from Ashlan, Inc.); diazolidinyl urea (e.g. Germall II Ashland, Inc.); and so forth. Combinations of one or more different preservatives are also suitable for use in the present invention. Desirably the preservative is present in the cleaning composition in an amount between about 0.001% and about 5% (by weight of the cleaning composition). In certain embodiments, the preservative may comprise between about 2.0% and about 0.01% by weight of the cleaning composition and, in still further embodiments, may comprise between about 1.0% and 0.1% by weight of the cleaning composition.

The liquid cleaning composition may, optionally, further include one or more skin benefit agents such as, for example, antioxidants, astringents, conditioners, emollients, external analgesics, film formers, humectants, pH modifiers, moisturizers, skin protectants, and so forth. Still further, additional agents may be added to achieve the desired viscosity, feel, or other attributes of the formulation as may be desired; for example, the cleaning composition can contain one or more rheology modifiers, slip agents, deodorants, fragrances, dyes and so forth.

### Wipes Dispensing

The wipes may be presented in a stacked format; the stack may, for example, include between 3 and 250 wet wipes and, in further embodiments may include between about 5 and about 150 wet wipes and in still further embodiments may include between about 8 and about 90 wet wipes. In one aspect, the sheets forming the stack may be superposed with one another in a folded or unfolded orientation. Various known folded configurations can be used in conjunction with the present invention including, for example, V-folds, Z-folds, W-folds, quarter-folds and so forth. In certain embodiments, the stack of wipes by be inter-leaved whereby the folded panel of one wipe is positioned between two folded panels of one or more adjacent wipes. Equipment and processes for forming dispensable stacks of wipes are known in the art; examples of which include, but are not limited to, those described in U.S. Pat. No. 3,401,927 to Frick et al.; U.S. Pat. No. 4,502,675 to Clark et al.; U.S. Pat. No. 5,310,398 to Yoneyama, U.S. Pat. No. 5,964,351 Zander, U.S. Pat. No. 6,612,462 to Sosalla et al., U.S. Pat. No. 6,991,840 to Sosalla et al., the contents of which are incorporated herein to the extent consistent herewith. In another aspect, such as with respect to product formats utilizing a continuous length of sheet material, the stack may be provided comprising individually separable wet wipes having perforated or over-bonded lines of weakness which allow ready separation into smaller individual sheets of a desired shape and size. In this regard, use of such lines of weakness are commonly employed in conjunction with stacks in a rolled format.

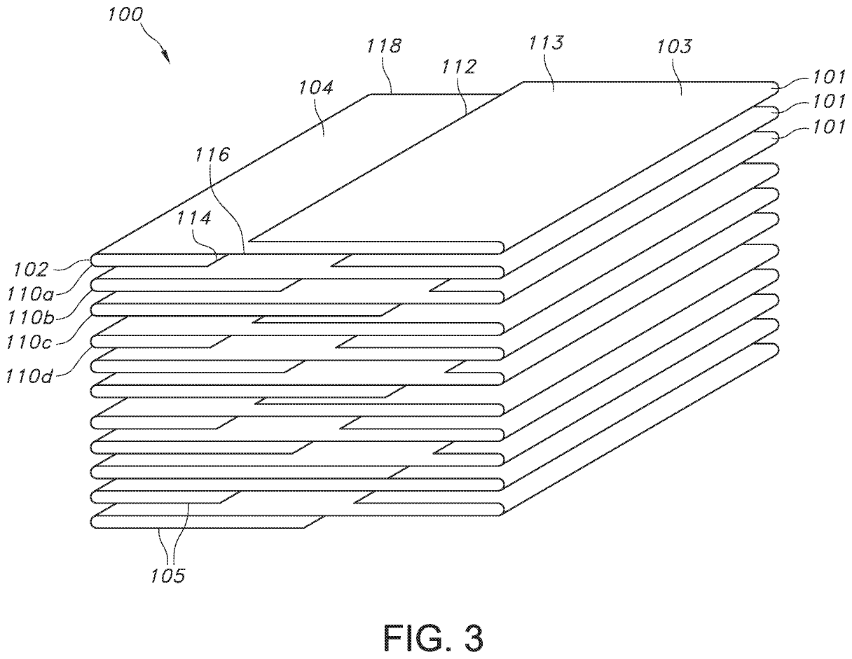

A stack is provided comprising a plurality of individual wipes; such individual wipes can be completely separated from one another or be inter-connected by separable elements such as frangible tabs as noted above. In reference to the embodiment shown in FIG. 3, a stack 100 includes plurality of superposed wipes 110 (110a, 110b, 110c, 110d). Each wipe 110 has first and second opposed edges 112, 114 and third and fourth opposed edges 116, 118. The first edge 112 is exposed on an outer surface of the stack 100 and in the present embodiment on the upper surface of the stack 100. In the present embodiment, the wipes 110 are presented in a z-fold format with each wipe having a first fold 101 proximate the first edge 112 forming a first outer panel 103 over a central panel 104 of the wipe 110 and a second opposite fold 102 proximate the opposed second edge 114 forming a second outer panel 105 folded under the central panel 104 of the wipe 110. The wipes 110 are sequentially folded and oriented such that removal of the outer or upper most wiper 110a will similarly present and expose the first edge 112 of the underlying wipe 110b in the stack 100. With respect to the formed stack, a cleaning formulation can be added thereto in a desired amount to form a stack of wet wipes.

The wet wipes can be maintained over time in a sealed container such as, for example, plastic pouches or bags, canisters, jars, tubs, buckets and so forth. When utilizing a stack of wet wipes the use of a resealable container is particularly desirable in order to limit evaporation of the cleaning composition from the remaining unused wet wipes in the container. Exemplary resealable containers and wet wipe dispensers include, but are not limited to, those described in U.S. Pat. No. 4,171,047 to Doyle et al., U.S. Pat. No. 4,353,480 to McFadyen, U.S. Pat. No. 4,778,048 to Kaspar et al., U.S. Pat. No. 4,741,944 to Jackson et al., and US2012/0160864 to Shoaf et al., the contents of which are incorporated herein to the extent consistent herewith. Flexible bag packaging with a resealable label are particularly well suited for use with the present invention and examples of the same include, but is not limited to, those described in U.S. Pat. No. 5,264,265 to Kaufmann, U.S. Pat. No. 6,592,004 to Huang et al., US2005/0011906 to Buck et al., US2010/0154264 to Scott et al., US2010/0155284 to Gerstle et al., and US2014/001196 to Bushman et al., the contents of which are incorporated herein to the extent consistent herewith. Dispensers employing the tabbed and/or continuous roll-type stacks are disclosed, for example, in U.S. Pat. No. 4,651,895 Niske et al., U.S. Pat. No. 6,158,614 to Haines et al., and US2010/0133287 to Tramontina et al., the contents of which are incorporated herein to the extent consistent herewith. The particular stack height and sheet count can vary with the intended format and use. The sheets can be oriented in the stack and the stack incorporated into the container in a manner intended to improve efficiency of use and/or dispensing as is known in the art. In certain embodiments, stacks of wet wipes are desirably arranged and combined with a dispenser to facilitate one at a time dispensing and including “pop-up” dispensing formats.

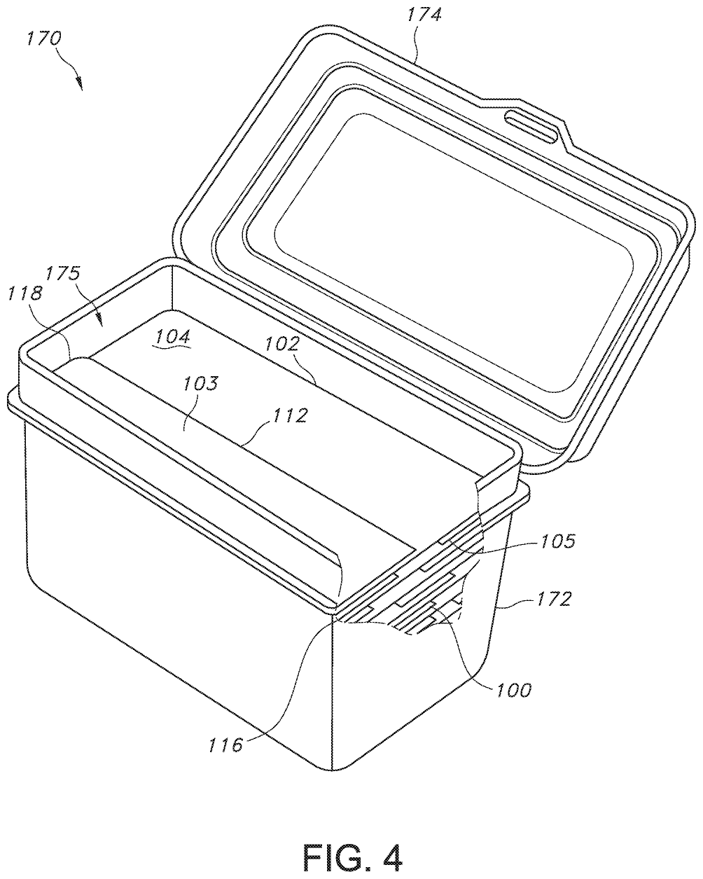

In one embodiment, and in reference to FIG. 4, a reach-in dispenser 170 is provided having a tub 172 and a reclosable lid 174 hingedly attached to the tub 172; the tub 172 and lid 174 cooperating to define a sealed or resealable interior 175 of the container 170. A stack of wipes 100 is positioned within the tub 172 wherein the first edge 112 is presented and exposed such that when the lid 174 is in an open state a user can reach in the tub, grasp the first edge 112 of the wipe 110 and remove the wipe 110 from the stack 100. Upon removing the upper most wipe, a subsequent wipe will be presented again with the first edge 112 being exposed for grasping by the user. Thereafter, the lid can be returned to a closed position (not shown) in order to prevent evaporative loss of the liquid cleaning composition contained in the wipes.

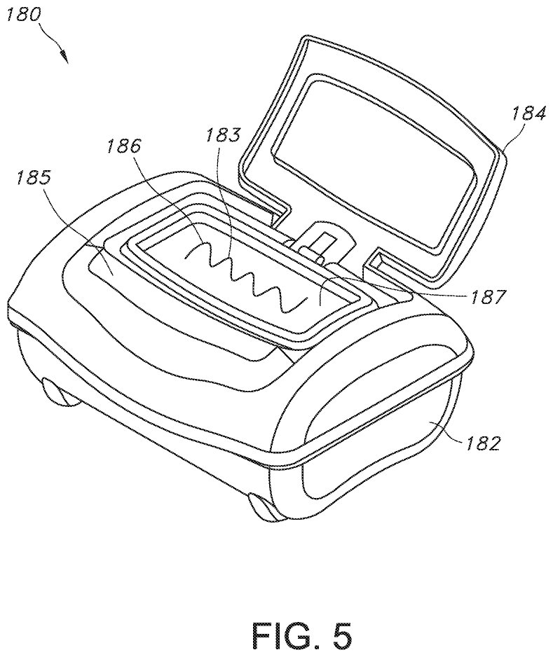

In certain embodiments, and in reference to FIG. 5, the dispenser 180 may also include a dispensing plate 185 intermediate to the tub 182 and lid 184. The dispenser 180 further includes a dispensing aperture 183 such as a series of one or more inter-connected slits 186. Alternatively, the dispensing aperture may comprise a defined opening (not shown). The dispensing aperture may be formed within a resilient or rubber-like film material 187 spanning an inner portion of the dispensing plate 185. When a wipe (not shown) is pulled through the dispensing aperture 183, the flexible film 187 acts to provide drag or frictional resistance such that when the leading wipe is fully pulled through the dispensing aperture and the leading edge of the subsequent wipe is pulled through the dispensing aperture, the drag acts to cause the inter-folded or inter-connected wipes to separate leaving the first edge of the next wipe above the stack and partially exposed and/or protruding through the dispensing aperture, thereby easily grasped by the user to provide one-at-a-time pop-up dispensing. Pop-up dispensing features such as this are known in the art and, by way of example only, are shown and described in U.S. Pat. No. 6,523,690 to Buck et al.; U.S. Pat. No. 6,585,131 to Huang et al. and U.S. Pat. No. 9,226,627 to Powling et al.

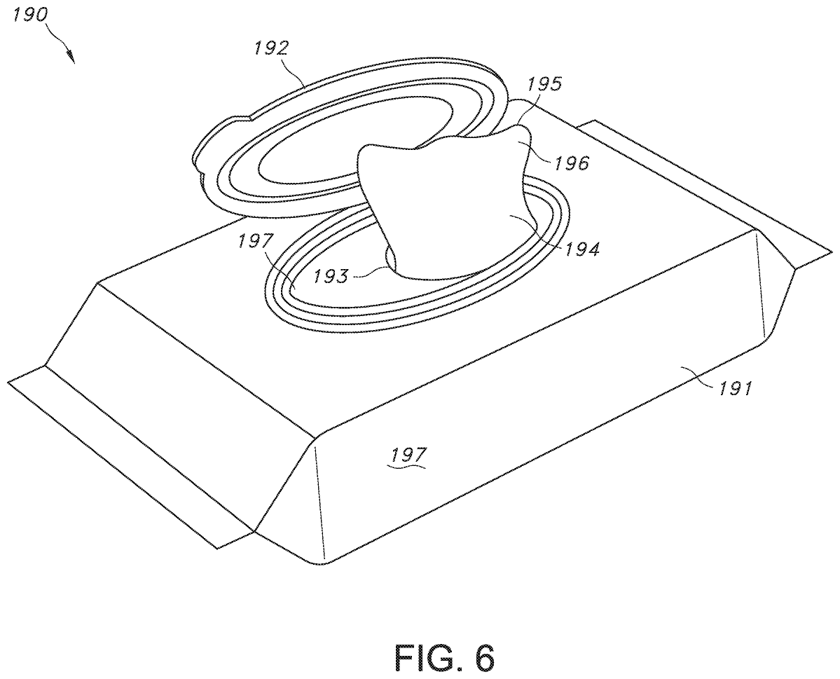

In a further embodiment and in reference to FIG. 6, a dispenser 190 is provided by a flexible pouch 191 and having thereon a rigid-resealable lid 192 attached to the flexible pouch 191 formed by a thin liquid impermeable material 197 such as a polyethylene film. As is known in the art, the flexible pouch includes a removable tab (not shown) such that when the user is ready to extract wipes, the removable tab is peeled away which exposes a dispensing orifice 193 through which a wipe 194 can be extracted and removed from the stack (not shown). The dispensing orifice 193 may, in certain embodiments, be defined by the flexible barrier material 197 that also forms the pouch, the edges of which may if desire be reinforced. Removal of a wipe from the stack via the dispensing orifice causes the underlying subsequent wipe (not shown) to present itself on the outside of the stack and be partially exposed through the dispensing orifice 193. The individual wipes are oriented such that the leading wiper edge 195 is presented for grasping, including in certain embodiments being presented in an elevated and exposed manner associated with one at a time, pop-up dispensing. The size of the dispensing orifice or port can be selected to achieve the desired dispensing function. In many embodiments, it will be desirable for the dispensing orifice to provide a constricted opening, namely an opening that is significantly smaller than that of the wipe itself. For example, the largest dimension of the dispensing opening may be less than 75% or even less than 50% of both the length and width of the wipe.

### Tests Methods

As used herein “basis weight” is determined using the bone dry weight of twelve (12) 150 mm×150 mm specimens.

The average fiber size of the meltblown fibers is determined by automated digital analysis of six

BSE/HICON surface SEM images. The image analysis software and system read the images, perform detection and image processing steps as well as acquire and calculate measurements. Additional information regarding the system and algorithm for conducting such measurements are described in greater detail in U.S. Pat. No. 8,017,534 to Harvey et al., the corresponding contents of which are incorporated herein by reference. However, in view of the nature of the coformed web, prior to taking the surface images used in the analysis, the absorbent staple length fibers are dissolved using a solvent that is inert with respect to the meltblown fibers. For example, a sulfuric acid bath can be used to remove cellulosic fibers while leaving the meltblown fibers intact.

The melt flow rate (“MFR”) as used herein means that measured in accordance with ASTM D1238-13, condition 230° C./2.16 kg using a melt indexer. With respect to determining MFR of the meltblown fibers, the absorbent fibers will be removed prior to conducting the analysis as noted above.

As used herein air permeability is conducted on bone dry samples and determined using a TEXTEST FX 3300 Air Permeability Tester from Textest AG using a test pressure of 125 Pa and a test head area of 38 cm².

As used herein “tensile strength” or “strip tensile”, is the peak load value, i.e. the maximum force produced by a specimen, when it is pulled to rupture. Samples for tensile strength testing are prepared by drying and then die cutting test specimens to a width of 25 mm and length of approximately 152 mm. The instrument used for measuring tensile strengths is an MTS Criterian 42 and MTS TestWorks™ for Windows Ver. 4 (MTS Systems Corp., Research Triangle Park, N.C.). The load cell is selected, depending on the strength of the sample being tested, such that the peak load values fall between 10 and 90 percent of the load cell's full scale load. The gauge length is 76 mm and jaw length is 76 mm. The crosshead speed is 305 mm/minute, and the break sensitivity is set at 70% and the slope preset points at 70 and 157 g. The sample is placed in the jaws of the instrument and centered with the longer dimension parallel to the direction of the load application. The test is then started and ends when the specimen breaks. The peak load is determined, for purposes herein, based upon the CD tensile strength. Six (6) representative specimens are tested, and the arithmetic average of all individual specimen tested is the tensile strength for the product.

Softness of the nonwoven webs were measured using an EMTEC Tissue Softness Analyzer (“TSA”) (Emtec Electronic GmbH, Leipzig, Germany) and in particular the TS7 value. The TSA comprises a rotor with vertical blades which rotate on the test piece applying a defined contact pressure. Contact between the vertical blades and the test piece creates vibrations, which are sensed by a vibration sensor. The sensor then transmits a signal to a PC for processing and display. The signal is displayed as a frequency spectrum. For measurement of TS7 values the blades are pressed against sample with a load of 100 mN and the rotational speed of the blades is 2 revolutions per second. To measure TS7, a frequency analysis is performed in the range from 1 to 10 kHZ, with the amplitude of the peak occurring at 7 kHz being recorded as the TS7 value. The TS7 value represents the softness of the sample and a lower amplitude correlates to a softer sample. In addition, test samples were prepared by cutting a circular sample having a diameter of 112.8 mm. All samples were allowed to equilibrate at TAPPI standard temperature and humidity conditions for at least 24 hours prior to completing the TSA testing. The sample is placed in the TSA with the air side of the sample facing upward (the wire side facing downward). The sample is secured and the measurements are started via the PC. The PC records, processes and stores all of the data according to standard TSA protocol. The reported values are the average of 12 replicates, each one with a new sample.

The Formation Index is calculated from optical images and data acquired using the Leica QWIN Pro software and the custom-written QUIPS routine ‘Formation—1 (nonwovens).’ As with other tests noted above, the non-meltblown fibers are removed in a non-destructive manner prior to analyzing the uniformity of the meltblown fibers.

The wipes can, optionally, include one or more additional elements or components as are known in the art. Thus, while the invention has been described in detail with respect to specific embodiments and/or examples thereof, it will be apparent to those skilled in the art that various alterations, modifications and other changes may be made to the invention without departing from the spirit and scope of the same. It is therefore intended that the claims cover or encompass all such modifications, alterations and/or changes.

What is claimed is:

1. A wipe comprising:
a nonwoven web having a first major outer surface and an opposed second major outer surface and an edge surface spanning said first and second major outer surfaces and wherein said nonwoven web comprises a mixture of polymeric meltblown fibers and absorbent staple length fibers;
said polymeric meltblown fibers comprising between about 20 wt. % and about 40 wt. % and said absorbent staple length fibers comprising between about 60 and about 80 wt. % of said nonwoven web;
said polymeric meltblown fibers having an average fiber size between 1.0 and 3.0 micrometers, wherein said polymeric meltblown fibers comprise a propylene polymer and have a melt-flow rate of between 200 and 950 dg/minute;
said nonwoven web having an air permeability between about 1.4 and about 8.5 m³/minute and a tensile strength of at least about 190 grams-force and wherein said polymeric meltblown fibers comprise the majority of fibers forming said first and second major outer surfaces and said absorbent staple length fibers comprise a minority of fibers forming the first and second major outer surfaces and further wherein said absorbent staple length fibers protrude from and/or are unoccluded by said polymeric meltblown fibers at said first and second major outer surfaces; and
a liquid cleaning formulation comprising water and a surfactant and further wherein said liquid cleaning formulation is present within said nonwoven web in an amount of from about 50% to about 700% based upon the weight of the dry nonwoven web

2. The wipe of claim 1 wherein said absorbent staple length fibers comprise cellulosic fibers.

3. The wipe of claim 2 wherein the nonwoven web has a normalized tensile strength of at least 15 g-f per g/m².

4. The wipe of claim 3 wherein said liquid cleaning composition comprises between about 70 to 99% water and from about 20% to about 0.5% surfactant.

5. The wipe of claim 3 wherein said meltblown fibers comprise between 20% and 35% of said nonwoven web and said staple length absorbent fibers comprise between 80% and 65% of said nonwoven web.

6. The wipe of claim 1 wherein said propylene polymer comprises at least about 85% of said polymeric meltblown fibers.

7. The wipe of claim 6 wherein said polymeric meltblown fibers have an MFR between about 250 and about 850 dg/minute.

8. The wipe of claim 7 wherein said meltblown fibers have an average fiber diameter between 2.9 and 1.5 micrometers.

9. The wipe of claim 8 wherein the first and second major outer surfaces of said nonwoven web have a TS7 softness rating of between about 4.5 and about 6.5 and further wherein said absorbent staple length fibers comprise wood pulp fibers.

10. The wipe of claim 1 wherein said nonwoven web has a pattern of embossments therein and wherein said embossments comprise less than about 30% of the area of said nonwoven web.

11. The wipe of claim 1 wherein the nonwoven web has a tensile strength between 220 and 400 grams-force and a basis weight between 30 and 90 g/m².

12. The wipe of claim 11 wherein said nonwoven web has an air permeability of between about 2 and about 7 m³/minute.

13. The wipe of claim 12 wherein said nonwoven web has a diameter, in its greatest dimension, of between about 5 and about 45 cm.

14. The wipe of claim 1 wherein said polymeric meltblown fibers are melt-extruded and formed from a propylene polymer having an MFR less than about 850 dg/minute.

15. The wipe of claim 14 wherein the first and second major outer surfaces of said nonwoven web have a TS7 softness rating of between about 5.0 and about 6.0.

16. The wipe of claim 14 wherein said nonwoven web has a Formation Index between 10.0 and 20.0.

17. A wipes dispenser comprising:
a resealable container having a dispensing opening and a reclosable lid;
a stack of nonwoven wipes contained within an interior of said container and presenting an outermost individual nonwoven wipe adjacent said dispensing opening;
said nonwoven wipes comprising the nonwoven wipe of claim 1;
wherein the edge of said outermost individual nonwoven wipe is positioned adjacent said dispensing opening.

18. The wipes dispenser of claim 17 wherein said dispenser has a constricted dispensing opening that is smaller than both the length and width of said wipe.

19. The dispenser of claim 17 wherein said dispenser further incudes a dispensing plate having a dispensing opening comprising an aperture defined within a flexible film.

20. The dispenser of claim 18 wherein said edge of the outermost wipe positioned adjacent the dispensing opening extends substantially in the machine direction.
